(19) United States
(12) Patent Application Publication
(10) Pub. No.: US 2023/0338274 A1
(43) Pub. Date: Oct. 26, 2023

(54) FOOD SUPPLEMENT FOR HAIR CARE AND HAIR LOSS PREVENTION.

(71) Applicant: DERMACISEN, S.A., Ciudad de Panama, PA (US)

(72) Inventor: Jose Luis Cisneros Vela, Barcelona (ES)

(21) Appl. No.: 17/727,473

(22) Filed: Apr. 22, 2022

(51) Int. Cl. A61K 8/9783; A61Q 7/00; A23L 33/15; A23L 33/105; A23L 33/16; A23L 33/175; A23L 33/18; A61K 8/34; A61K 8/27; A61K 8/20; A61K 8/67; A61K 8/65; A61K 8/36

(52) U.S. Cl. CPC A61K 8/9783; A61Q 7/00; A23L 33/15; A23L 33/105; A23L 33/16; A23L 33/175; A23L 33/18; A61K 8/347; A61K 8/27; A61K 8/20; A61K 8/673; A61K 8/676; A61K 8/678; A61K 8/65; A61K 8/36

(57) ABSTRACT

The invention relates to a food supplement for hair care and hair loss prevention, comprising capsules for oral administration which are intended for human consumption and contain components of the food supplement. Said food supplement includes the following components and percentages by weight with respect to the total weight of the food supplement: between 75% and 80% active ingredients, between 5% and 8% anti-caking and stabilizing additives, and between 12% and 20% a cover forming the outside of the capsule.

### TECHNICAL FIELD

The present invention relates to a food supplement for oral administration intended for human consumption and the main purpose of which is hair care and hair loss prevention.

### PRIOR ART

Society today places a special importance on people's image, and certain factors such as alopecia have a negative influence on a person's appearance; therefore, there is great personal interest in hair care and hair loss prevention.

In the field of cosmetics, there are a number of body and hair care products consisting primarily of creams and lotions for topical use.

While these creams or lotions for topical use provide a moisturizing effect for the skin and hair that may contribute to delaying or minimizing wrinkles, but they have a very limited effect in treating hair loss, due precisely to their topical or external application, which prevents them from acting on factors that are significantly involved in hair loss and answer to internal causes, whether of the androgenic type or due to a deficiency in vitamins or nutrients suitable for strengthening hair and preventing hair loss.

Manufacturers of body care products, who are aware of this need, are particularly interested in developing products to fight against hair loss, so there are different products on the market as well in the form of tablets or liquids for oral consumption, which are intended for hair care and the composition of which mainly includes a vitamin or a specific active ingredient to achieve a certain effect.

Given that diverse factors of both the androgenic type and due to food may affect hair loss, the consumption of said products may have a lower or no effect in different people, depending on whether or not the vitamin or active ingredient included in the product is suitable for treating the factor or factors which cause hair loss in each person and which, among others, may be of an androgenic, hormonal, food, or nervous origin.

Therefore, the problem considered is the development of a food supplement in the form of capsules for oral administration intended for human consumption, developed specifically for hair care and hair loss prevention and acting simultaneously on different factors causing hair loss which, as indicated, may be of an androgenic type, due to a deficiency of vitamins, minerals, or active ingredients necessary for strengthening hair and preventing hair loss, or of a nervous type.

The applicant of the invention is unaware of the existence in the state of the art of food supplements for hair care and hair loss prevention with the composition and the properties of the food supplement of the present invention.

### DISCLOSURE OF THE INVENTION

The food supplement for hair care and hair loss prevention, object of the invention, comprises capsules for oral administration which are intended for human consumption and contain components acting jointly and synergistically on different factors which may negatively affect hair nutrition and conservation; said food supplement providing a supply of active ingredients, making it particularly indicated for hair care and hair loss prevention.

This food supplement is presented in the form of capsules for oral administration for human consumption, including the following components and proportions by weight, with respect to the total weight of the food supplement.
between 75% and 80% active ingredients.
between 5% and 8% anti-caking and stabilizing additives.
between 12% and 20% a cover forming the outside of the capsule.

Namely and according to the invention, the active ingredients and the percentages by weight thereof with respect to the total weight of the food supplement are itemized below:
between 15% and 20% bamboo steam dry extract (Bambusa arundinacea);
between 15% and 20% L-Cystine;
between 6% and 9% red clover aerial part dry extract;
between 5% and 7% Pygeum africanum dry bark extract;
between 5% and 7% keratin;
between 5% and 7% Sabal serrulata dry fruit extract;
between 4.5% and 6.5% Ferrous Fumarate;
between 1% and 3% zinc sulfate 1-hydrate;
between 1% and 3% L-ascorbic acid (Vitamin C);
between 1.5% and 2.5% D-pantothenate, calcium (Pantothenic acid-Vitamin B5);
between 1% and 3% Polygonum cuspidatum root (95% Resveratrol)
between 0.5% and 0.80% DL·alpha tocopherol acetate (vitamin E);
between 0.5% and 0.80% Lycopene 10% (Lycopersicum Esculentum Fruit);
between 0.1% and 0.5% Pyridoxine hydrochloride (Vitamin B6);
between 0.1% and 0.5% Cyanocobalamin 0.1% (Vitamin B12);
between 0.01% and 0.05% Sodium selenite; between 0.008% and 0.03% D-biotin (Biotin) vitamins H, B7, and B8.

The mentioned active ingredients have specific properties with different treating functions, namely:
D-Alpha Tocopherol acetate, L-ascorbic acid, D-Pantothenate, calcium (Pantothenic acid), pyridoxine hydrochloride, cyanocobalamin, and D-biotin supply the body with vitamins E,
H, B5, B6, B7, B8, and B12, with antioxidant properties, relating to the protection of the immune, nervous, and cardiovascular systems, and with cell growth;
Bamboo steam dry extract (Bambusa arundinacea) is a constituent of keratin and plays a favorable role in metabolism in skin and hair;
Red clover aerial part dry extract acts on hormonal dysfunction and protein loss of the extracellular matrix;
Pygeum africanum dry bark extract is an inhibitor of the 5-alpha-reductase enzyme, which transforms testosterone into dihydrotestosterone which is responsible for baldness.
Keratin is a protein that participates in hair tissue formation;
Sabal serrulata dry fruit extract stops hair loss;
Ferrous Fumarate is an anti-anemia iron supplement;
Zinc sulfate 1-hydrate is a dietary product which favors rehydration;
Polygonum cuspidatum root, with a high content of resveratrol, and lycopene have a very positive action as antioxidants; and
Sodium selenite regulates thyroid function by collaborating in keeping hair in good condition.

The combination of these active ingredients produces a synergistic effect in hair treatment and hair loss prevention, given that they act simultaneously on different factors causing hair loss in both men and women.

According to the invention, the anti-caking and stabilizing additives include the following components and ranges of percentages by weight, with respect to the total weight of the product:
between 6% and 8% microcrystalline cellulose
between 1% and 3% magnesium stearate vegetable
between 0.1% and 1% silicon dioxide (E551b)

Microcrystalline cellulose is a bulking agent acting as a stabilizing agent, while magnesium stearate and silicon dioxide act as binders.

The aforementioned components are contained in a transparent vegetable capsule formed by a cover, namely hydroxypropyl methylcellulose, the weight of the capsule constituting between 13% and 17% of the total weight of the food supplement.

### DETAILED DISCLOSURE OF EMBODIMENTS OF THE INVENTION

In a specific exemplary embodiment, the food supplement for hair care and hair loss prevention consists of capsules weighing 800 mg, in respect of which capsules: 677 mg correspond to the active ingredients and additives included inside the capsule, and the remaining 123 mg correspond to the capsule or outer shell.

In this specific example, the food supplement comprises the following composition of: active ingredients, additives, and outer cover, with the percentages by weight indicated with respect to the total weight of the food supplement:

| | Weight | Weight |
|---|---|---|
| Component | (mg) | (%) |
| Active ingredients: | | |
| DL•alpha tocopherol acetate | 5.00 | 0.63% |
| (50% Vitamin E) | | |
| Bamboo steam dry extract | 142.86 | 17.86% |
| (Bambusa arundinacea) | | |
| L-Cystine | 150.00 | 18.75% |
| Red clover aerial part dry extract | 63.00 | 7.88% |
| Pygeum africanum dry bark extract | 50.00 | 6.25% |
| Keratin | 50.00 | 6.25% |
| Sabal Serrulata dry fruit extract | 50.00 | 6.25% |
| Ferrous Fumarate | 42.55 | 5.32% |
| Zinc sulfate 1-hydrate | 16.05 | 2.01% |
| L-ascorbic acid (Vitamin C) | 15.00 | 1.88% |
| D-Pantothenate, calcium | 10.87 | 1.36% |
| (Pantothenic acid-Vitamin 85) | | |
| Polygonum cuspidatum root | 9.47 | 1.18% |
| (95% Resveratrol) | | |
| Lycopene 10% | 5.00 | 0.63% |
| Pyridoxine hydrochloride (Vitamin B6) | 2.43 | 0.30% |
| Cyanocobalamin 0.1% (Vitamin B12) | 1.00 | 0.13% |
| Sodium selenite | 0.12 | 0.02% |
| D-biotin (Biotin) (Vitamins H, B7, and B8) | 0.05 | 0.01% |
| Additives: | | |
| Microcrystalline cellulose | 53.60 | 6.70% |
| Magnesium stearate vegetable | 6.00 | 0.75% |
| Silicon dioxide | 4.00 | 0.50% |
| Cover (Capsule): | | |
| Transparent vegetable capsule | 123.00 | 15.38% |
| (Hydroxypropyl methylcellulose) | | |
| TOTAL | 800.00 | 100.00% |

Having sufficiently described the nature of the invention as well as a preferred embodiment thereof, it is hereby stated for all relevant purposes that the materials, shape, size, and arrangement of the described elements may be modified, provided that this does not entail an alteration of the essential features of the invention claimed below.

1. A food supplement for hair care and hair loss prevention, object of the invention, comprises capsules for oral administration which are intended for human consumption and contain components of the food supplement; wherein said food supplement includes the following components and percentages by weight with respect to the total weight of the food supplement:
between 75% and 80% active ingredients;
between 5% and 8% anti-caking and stabilizing additives; and
between 12% and 20% a cover forming the outside of the capsule.

2. The food supplement according to claim 1, wherein the active ingredients comprise the following components and the percentages by weight thereof with respect to the total weight of the food supplement:
between 15% and 20% Bamboo steam dry extract (Bambusa arundinacea);
between 15% and 20% L-Cystine;
between 6% and 9% Red clover aerial part dry extract;
between 5% and 7% Pygeum africanum dry bark extract;
between 5% and 7% keratin;
between 5% and 7% Sabal serrulata dry fruit extract;
between 4.5% and 6.5% Ferrous Fumarate;
between 1% and 3% zinc sulfate 1-hydrate;
between 1% and 3% L-ascorbic acid (Vitamin C);
between 1.5% and 2.5% D-pantothenate, calcium (Pantothenic acid-Vitamin B5);
between 1% and 3% Polygonum cuspidatum root (95% Resveratrol);
between 0.5% and 0.80% DL·alpha tocopherol acetate (vitamin E);
between 0.5% and 0.80% Lycopene 10% (Lycopersicum Esculentum Fruit);
between 0.1% and 0.5% Pyridoxine hydrochloride (Vitamin B6);
between 0.1% and 0.5% Cyanocobalamin 0.1% (Vitamin B12);
between 0.01% and 0.05% Sodium selenite; and
between 0.008% and 0.03% D-biotin (Biotin) vitamins H, B7, and B8.

3. The food supplement according to claim 1, wherein the anti-caking and stabilizing additives comprise:
between 6% and 8% microcrystalline cellulose;
between 1% and 3% magnesium stearate vegetable; and
between 0.1% and 1% Silicon dioxide (E551b).

4. The food supplement according to claim 1, wherein the cover forming the outside of the capsule is hydroxypropyl methylcellulose.
